(19) United States
(12) Patent Application Publication
(10) Pub. No.: US 2014/0128689 A1
(43) Pub. Date: May 8, 2014

(54) JOINT SENSOR DEVICES AND METHODS

(71) Applicants: Kirk W. Stewart, Woburn, MA (US); Noah Pestana, Roxbury, MA (US); Nicholas Quigley, Brighton, MA (US); Chris Proulx, Manchester, NH (US)

(72) Inventors: Kirk W. Stewart, Woburn, MA (US); Noah Pestana, Roxbury, MA (US); Nicholas Quigley, Brighton, MA (US); Chris Proulx, Manchester, NH (US)

(73) Assignee: NORTHEASTERN UNIVERSITY, Boston, MA (US)

(21) Appl. No.: 14/110,003

(22) PCT Filed: Apr. 6, 2012
(86) PCT No.: PCT/US12/32534

§ 371 Date: Jan. 17, 2014

Related U.S. Application Data
(60) Provisional application No. 61/472,350, filed on Apr. 6, 2011.

(51) Int. Cl. A61B 5/00; A61B 7/00; A61B 5/01; A61F 5/01; A61B 5/11

(52) U.S. Cl. CPC A61B 5/4528; A61F 5/0102; A61B 5/1121; A61B 5/01; A61B 5/6828; A61B 5/0008; A61B 5/742; A61B 5/7246; A61B 5/7282; A61B 7/006
USPC 600/301

(57) ABSTRACT

Joint sensor devices, bracing members, and methods of use are provided. A joint sensor device includes a bracing member contoured to fit over a joint on a limb of a human subject, such that the bracing member allows the limb to pivot; at least a first sensor operably connected to the bracing member, such that the first sensor is configured to detect an angle of the joint; at least a second sensor operably connected to the bracing member, such that the second sensor is configured to detect a vibration caused by joint movement; and a data communication device operably connected to the sensors, wherein the data communication device communicates signals from the sensors to an external device. The devices can also include temperature sensors. Bracing members and methods of monitoring joint health of subjects are also disclosed.

### CROSS REFERENCE TO RELATED APPLICATIONS

This application claims the benefit of U.S. Provisional Application No. 61/472,350, filed Apr. 6, 2011, the entire contents of which are hereby incorporated by reference herein.

### INCORPORATION BY REFERENCE

All patents, patent applications and publications cited herein are hereby incorporated by reference in their entirety in order to more fully describe the state of the art as known to those skilled therein as of the date of the invention described herein.

### BACKGROUND OF THE INVENTION

The technology described herein relates to joint sensor devices, bracing members, and methods for their use.

Arthropathy, or joint disease, includes various forms of arthritis, such as bacterial arthritis, osteoarthritis, or rheumatoid arthritis, and results from infection, trauma, degenerative changes, autoimmune diseases, and other causes. Human error can affect a medical professional's ability to accurately diagnose and treat arthropathy. Poor patient memory, poor patient records, and miscommunications between the patient and the medical professional during an interview can result in diagnosis errors. Patients sometimes do not report joint problems because they may not experience pain, and symptoms can be sporadic. In the case of degenerative and chronic diseases, this delay in diagnosis and thus treatment can result in permanent joint damage.

Conventional methods to diagnose joint health have drawbacks. Joint health is examined either non-invasively through magnetic resonance imaging (MRI) or computed tomography (CT) scans, or invasively through an arthroscopy. The non-invasive techniques are expensive and do not provide enough resolution to detect early or minimal joint damage. These methods can involve injecting the patient with a contrast material, such as a dye, which can have adverse side effects. Arthroscopy is a costly surgical procedure that involves insertion of an endoscope into the joint interior, and has several drawbacks. Because the medical professional cannot accurately diagnose a condition before the procedure, the most effective method of treatment cannot be discussed with the patient until after arthroscopy. Also, inherent risks are associated with an invasive procedure, including the possibility of infection, clotting, and permanent nerve injury.

Therefore, a need exists in the art for devices and methods to monitor joint health and to accurately and timely diagnose arthropathy.

### SUMMARY OF THE INVENTION

This disclosure describes joint sensor devices, bracing members, and methods for their use. The disclosed devices and methods are useful in various fields, including rehabilitation, primary care, sports medicine, elderly care, and personalized medicine.

In one aspect, the disclosed joint sensor devices comprise (a) a bracing member contoured to fit over a joint on a limb of a subject, wherein the bracing member allows the limb to pivot; (b) at least a first sensor operably connected to the bracing member, wherein the first sensor is configured to detect an angle of the joint; (c) at least a second sensor operably connected to the bracing member, wherein the second sensor is configured to detect a vibration caused by joint movement; and (d) a data communication device operably connected to the sensors, wherein the data communication device communicates signals from the sensors to an external device.

In one or more embodiments, the bracing member acts as the first sensor. In one or more embodiments, the devices further comprise at least a third sensor operably connected to the bracing member, wherein the third sensor is configured to detect a temperature. In one or more embodiments, the third sensor is configured to detect the temperature of the joint and/or of a location away from the joint.

In one or more embodiments, the bracing member is a sleeve having an inner surface and an outer surface.

In one or more embodiments, the data communication device communicates sensor signals to an external device using a wireless communication protocol. In one or more embodiments, the data communication device communicates sensor signals to an external device using a wire. In one or more embodiments, the external device comprises memory and is configured to store and display the sensor signal data.

In one or more embodiments, the second sensor includes a sensing tip, wherein the tip increases detection sensitivity. In one or more embodiments, the limb is the knee of the subject.

In one aspect, bracing members comprise (a) a flexible member contoured to fit over a joint on a limb of a subject, wherein the flexible member allows the limb to pivot; (b) at least a first sensor operably connected to the flexible member, wherein the first sensor is configured to detect an angle of the joint; and (c) at least a second sensor operably connected to the flexible member, wherein the second sensor is configured to detect a vibration caused by joint movement.

In one or more embodiments, the flexible member acts as the first sensor. In one or more embodiments, the bracing member further comprises at least a third sensor operably connected to the flexible member, wherein the third sensor is configured to detect a temperature.

In one or more embodiments, the third sensor is configured to detect the temperature of the joint and/or of a location away from the joint.

In one or more embodiments, the flexible member is a sleeve having an inner surface and an outer surface.

In one or more embodiments, the second sensor includes a sensing tip, wherein the tip increases detection sensitivity.

In another aspect, the disclose methods of monitoring joint health of a subject comprise (a) obtaining data using the joint sensor device of any one of claims 1 to 10, wherein the angle and vibration data are correlated; (b) establishing a baseline data range over a period of time that reflects typical functioning of the joint of a subject; and (c) comparing the baseline data range to test data from the subject to monitor joint health.

In one or more embodiments, the temperature data are correlated with the angle and vibration data in step (a). In one or more embodiments, the methods further comprise alerting the subject when the test data is significantly different from the baseline data range. In one or more embodiments, the methods further comprise diagnosing an arthropathy based on the comparison of the baseline data range to the test data.

### BRIEF DESCRIPTION OF THE DRAWINGS

The foregoing and other objects of the present invention, the various features thereof, as well as the invention itself, may be more fully understood from the following description, when read together with the accompanying drawings. The drawings are presented for the purpose of illustration only and are not intended to limit the invention.

FIG. 1 is a schematic diagram illustrating aspects of one embodiment of the disclosed devices.

FIG. 2 is a schematic diagram illustrating aspects of another embodiment of the disclosed devices.

FIG. 3 is a schematic diagram of piezoelectric sensors used in the disclosed devices, according to one or more embodiments.

FIG. 4 is a schematic diagram of thermistors and a goniometer used in the disclosed devices, according to one or more embodiments.

FIG. 5 is a graphical representation of data generated using an embodiment of the disclosed device.

FIG. 6 is a graphical representation of data generated using an embodiment of the disclosed device.

FIG. 7 is a graphical representation of vibration versus time data generated for two joints using one embodiment of the disclosed device.

FIG. 8 is a graphical representation of vibration versus time data generated for two joints using one embodiment of the disclosed device.

FIG. 9 is a schematic diagram illustrating aspects of one embodiment of the disclosed devices.

FIGS. 10A-D are schematic diagrams of the graphical user interface used with the disclosed devices and methods, according to one or more embodiments.

FIGS. 11A-B are schematic diagrams of the graphical user interface used with the disclosed devices and methods, according to one or more embodiments.

FIG. 12 is a schematic diagram illustrating one embodiment of the disclosed devices.

FIG. 13 is a schematic diagram illustrating one embodiment of the disclosed devices.

FIG. 14A-B are graphical representations of unfiltered and filtered vibration data, respectively, generated using one embodiment of the disclosed device.

### DETAILED DESCRIPTION

The disclosed devices and methods could be used in the fields of rehabilitation, primary care, sports medicine, elderly care, and personalized medicine. Thus, the embodiments of the disclosed devices and methods can be used to monitor joint conditions of the elbow, shoulder, torso, wrist, finger, ankle, hips, and knee. The joint sensor devices can be used in various configurations for various joints, including articulations of hand, elbow joints, wrist joints, axillary articulations, sternoclavicular joints, vertebral articulations, temporomandibular joints, sacroiliac joints, hip joints, articulations of foot, and knee joints.

### Joint Sensor Devices

In one aspect, the disclosure includes joint sensor devices. In one aspect, the joint sensor devices are used for a joint of the elbow, shoulder, torso, wrist, finger, ankle, hips, or knee. In another aspect, the joint sensor devices include a bracing member contoured to fit over a joint on a limb of a subject, wherein the bracing member allows the limb to pivot; at least a first sensor operably connected to the bracing member, wherein the first sensor is configured to detect an angle of the joint; at least a second sensor operably connected to the bracing member, wherein the second sensor is configured to detect a vibration caused by joint movement; and a data communication device operably connected to the sensors, wherein the data communication device communicates signals from the sensors to an external device. In some embodiments, the angle of the joint is defined by the angle formed by the two lines that correspond to the longitudinal axes of the bones that are joined.

In some embodiments, the bracing member of the joint sensor devices acts as the first sensor that detects an angle of the joint. In other embodiments, the joint sensor devices also include a third sensor that detects the temperature of the joint and/or a location away from the joint. The temperature of the joint is the surface temperature of the skin covering the joint, which is correlated to the internal temperature of the joint. The temperature measurement of the location away from the joint presents a reference temperature for a surface of the skin that is not affected by, for example, inflammation of the joint.

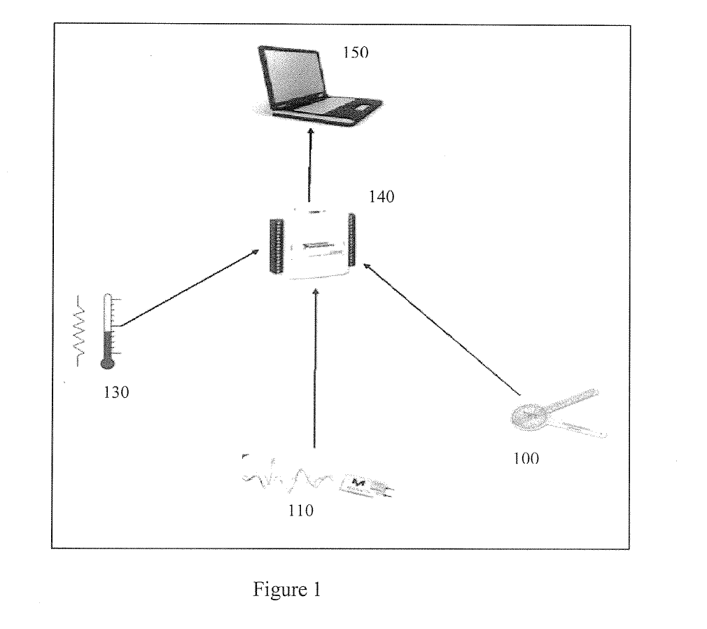

FIG. 1 is a schematic diagram illustrating aspects of one embodiment of the disclosed devices. FIG. 1 illustrates a first sensor 100 that detects angle measurements (for example, a goniometer), a second sensor 110 that detects vibrations caused by joint movement, and a third sensor 130 that detects temperature measurements. The sensors are operably connected to a data communication device 140, which communicates signals from the sensors to an external device 150. In one or more embodiments, the external device comprises memory and is configured to store and display the sensor signal data.

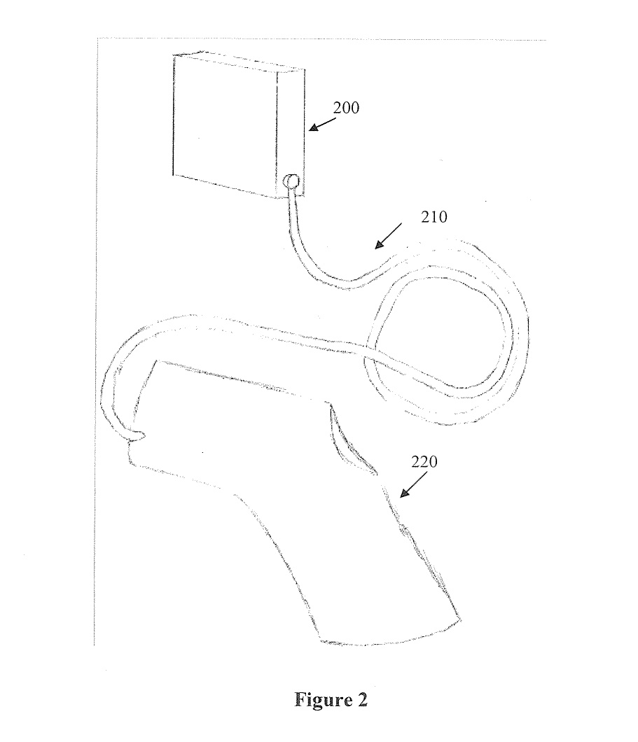

In one or more embodiments, the bracing member is a sleeve that has an inner surface and an outer surface. FIG. 2 is a schematic diagram illustrating aspects of another embodiment of the disclosed devices. A data communication device 200 is connected via a wire 210 to the bracing member, which is a sleeve 220, having an inner surface and an outer surface. Vibration, angle, and temperature sensors (not shown in FIG. 2) are connected to the sleeve 220. In one or more embodiments, the external device communicates sensor signals to an external device using a wireless communication protocol.

In one or more embodiments, the vibration, angle, and temperature sensors are detachable and replaceable. In some embodiments, for each new test, new sensors can be used. Detachable sensors are more hygienic and decrease wear and tear of the disclosed devices.

### Bracing Member

In one aspect, the bracing member is contoured to fit over a joint of the elbow, shoulder, torso, wrist, finger, ankle, hips, or knee, and the like.

In one aspect, bracing members include a flexible member contoured to fit over a joint on a limb of a subject, wherein the bracing member allows the limb to pivot; at least a first sensor operably connected to the bracing member, wherein the first sensor is configured to detect an angle of the joint; and at least a second sensor operably connected to the bracing member, wherein the second sensor is configured to detect a vibration caused by joint movement. In one or more embodiments, the bracing member acts as the first sensor that is configured to detect an angle of the joint. In one or more embodiments, the bracing member comprises a third sensor operably connected to the bracing member, wherein the third sensor is configured to detect a temperature. In one or more embodiments, the third sensor is configured to detect the temperature of the joint and/or the skin covering the joint.

In one or more embodiments, the subject is a vertebrate. In one or more embodiments, the subject is a mammal. In one or more embodiments, the subject is a human.

In one or more embodiments, the flexible member is a sleeve having an inner surface and an outer surface, as depicted in flexible member 220 in FIG. 2.

In one or more embodiments, the bracing member can be made of a rigid, semi-rigid, or flexible material. The bracing member can be made from a variety of materials, including but not limited to plastic, metal, fiber materials, such as carbon fiber, foam, elastic materials, and a combination thereof.

In one or more embodiments, the flexible member is contoured to fit over a joint on a limb of the subject is attached to the limb using a variety of attachment elements, including but not limited to straps, buttons, fasteners, connectors, tapes, adhesives, and other interlocking fabric materials, such as Velcro.

In one or more embodiments, the bracing member includes a polycentric hinge or an offset hinge that allows the limb to pivot.

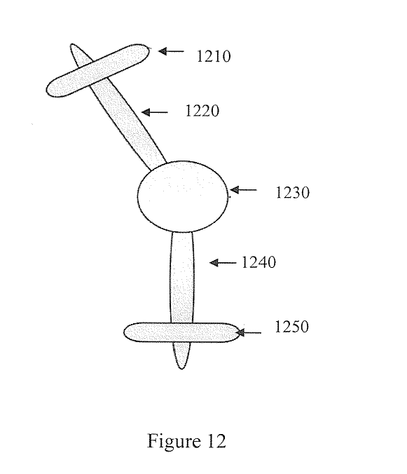

In one or more embodiments, the data communication device is mounted on a strap around the upper extremity of the limb. In one or more embodiments, the goniometer is mounted between the upper limb strap and lower limb strap. FIG. 12 is a schematic diagram illustrating one embodiment of the disclosed devices. The data communication device is mounted on strap 1210 around the upper limb 1220. The lower limb strap 1250 is secured around the lower limb 1240. The goniometer 1230 is mounted between the upper limb 1220 and lower limb 1240 and acts as the bracing member. In one or more embodiments, the limb is the leg. In one or more embodiments, the limb is the arm.

### Data Communication Device

The disclosed data communication device can also be referred to as a data acquisition system (DAS or DAQ), and can convert analog waveforms into digital values for processing. In one or more embodiments, the data communication device is operably connected to the sensors, and the sensors convert physical parameters to electric signals. In one or more embodiments, the DAQ has signal conditioning circuitry that converts sensor signals into a form that can be converted to digital values. In one or more embodiments, the DAQ has an analog-to-digital converter that converts the sensor signals into digital values. In one or more embodiments, the data communication device and power source are located within an electronics encasing, and the sensors exit the encasing via a wire that attach to the joint on a limb of the subject.

In one or more embodiments, the National Instruments (NI) USB-6008 DAQ is used as the data communication device that is operably connected to the sensors. This device has twelve digital input/output (DIO) channels, two analog output (AO) channels, eight analog input (AI) channels, and a 32-bit counter with USB interface. In one or more embodiments, the DAQ reads in the data from the temperature and vibration sensors and outputs it to a personal computer (PC) through a USB connection. In some embodiments, the external device is a PC that comprises memory and is configured to store and display the sensor signal data.

In one or more embodiments, the Nl USB-6008 DAQ allows for multiple readings to be taken simultaneously through its digital and analog I/O channels. The DAQ samples the vibration sensor, temperature sensor, and electro-goniometer at a rate of 300 samples per second for 3000 samples in a 10 second trace through multiple analog input channels. These samples are then processed in LabVIEW software and the results are written to a .csv file to be used by the software package. In one or more embodiments, the DAQ is supplied by the +/−9V battery power source and has a specified +5V digital output. In one or more embodiments, the actual measured output voltage of the DAQ was found to be 5.46V. Any DAQ that can interface with an external device can be used in the instant disclosure.

In one aspect, the joint sensor device includes (a) a bracing member contoured to fit over a joint on a limb of a subject, wherein the bracing member allows the limb to pivot; (b) at least a first sensor operably connected to the bracing member, wherein the first sensor is configured to detect an angle of the joint; (c) at least a second sensor operably connected to the bracing member, wherein the second sensor is configured to detect a vibration caused by joint movement; and (d) a data communication device operably connected to the sensors, wherein the data communication device communicates signals from the sensors to an external device. In one or more embodiments, the devices further includes at least a third sensor operably connected to the bracing member, wherein the third sensor is configured to detect a temperature.

### Power Source

In some embodiment, the power source can be standard, 9V Energizer batteries. These batteries were modified by connecting the positive lead of one battery to the negative lead of the other battery, which effectively supplies the system with both a positive 9V and a negative 9V with respect to ground. These batteries are easy to change, and are lightweight, so they can easily be mounted inside of the electronics box. The device can also include connections to a power source, for example, through electrical cables or cords.

### Vibration Sensors

Vibration data can indicate possible damage to the joint of the subject. Any vibration sensor capable of sensing vibrations over the joint are contemplated. In one or more embodiments, the vibration sensor is a piezoelectric film transducer. In one or more embodiments, the vibration sensor is similar to a seismometer. Exemplary vibration sensors are LDTO Solid State Switch/Vibration Sensors from Measurement Specialties Incorporated. Piezoelectric film transducers can be used to sense vibrations generated from joint movement. The vibration sensor creates a voltage when the shape of a flexible end of the sensor is changed, for example, when there is bending or vibration of the flexible end. In one or more embodiments, the piezoelectric sensor has a flexible portion in the middle of the sensor with both distal ends of the sensor in contact, either directly or indirectly, with the limb. These vibration sensors require no power input for operation, and the output is a voltage that is proportional to the frequency of vibration that is present in the joint.

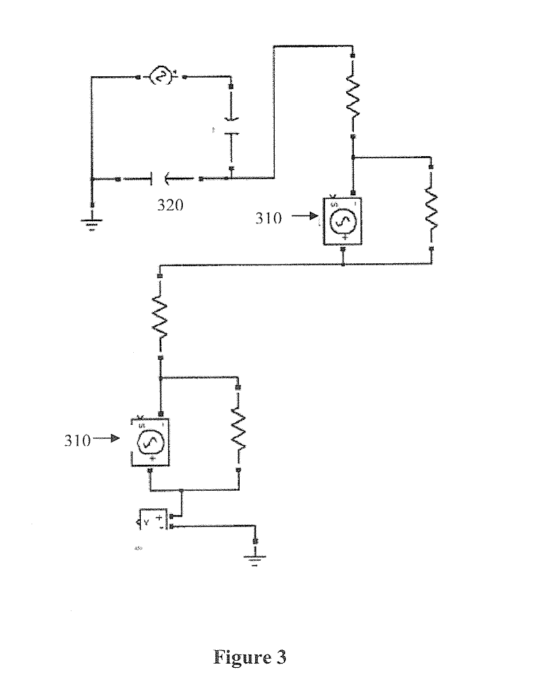

FIG. 3 is a schematic diagram of an exemplary piezoelectric sensor used in the disclosed devices. The output of the vibration sensor is an analog signal that is the same frequency as the physical stimulation applied to it. In some embodiments, the amplitude of the output is 50 mV per gram of force applied to the flexible end of the sensor. The vibration sensor signal is stepped up through two inverting op-amps 310 and 320 to amplify the signal before it is sent to the DAQ. Each op-amp is configured for a gain of 10. Noise-reducing capacitors 320 (220 μF) help reduce the power noise in the system. Various numbers of noise-reducing capacitors are contemplated, ranging from 1, 2, 3, 4, 5, 6, 7, 8, 9, to 10, etc. The gain stages amplify the signal before it arrives at the DAQ to differentiate the signal from inherent noise in the system. The signal is finally processed in the DAQ using LabVIEW software through an exponential averaging filter with alpha=0.03 to smooth out the signal, which is recorded as a function of voltage versus time. The filtered signal can be calculated as follows:

1. First point of filtered signal is set to the same value as the original signal.

2. Each subsequent point of the filtered signal is calculated as:
a. Filtered_signal(n)=a*filter_signal(n−1)+(a−1)*original signal
b. Where n is the index of the signal and a is a constant between 0 and 1
c. alpha was chosen to be 0.03 to smooth out the signal.

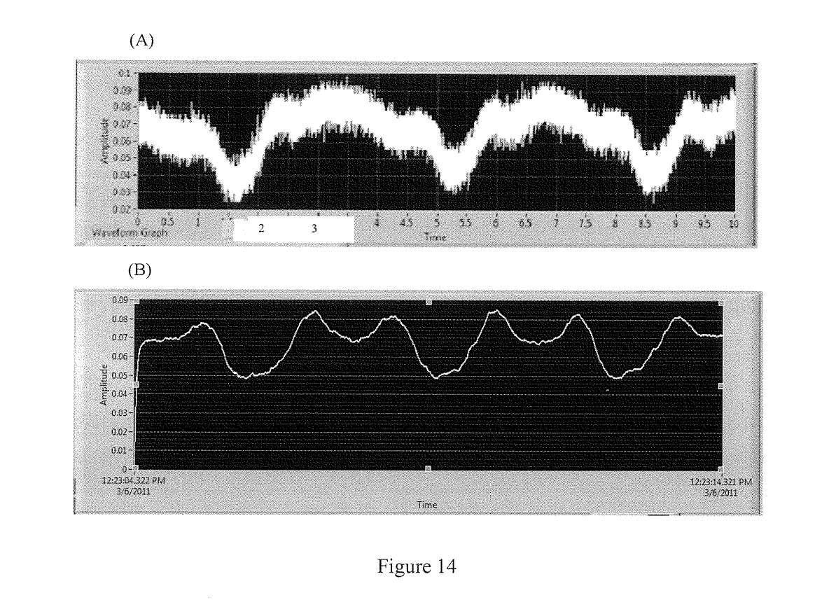

In FIG. 14A-B are graphical representations of unfiltered and filtered vibration data generated using one embodiment of the disclosed device. FIG. 14A represents raw vibration data that was sent directly to the DAQ. FIG. 14B represents vibration data that was processed using exponential averaging smoothing filter to reduce noise in the data. The x-axis represents time (seconds), and the y-axis represents amplitude of the vibration.

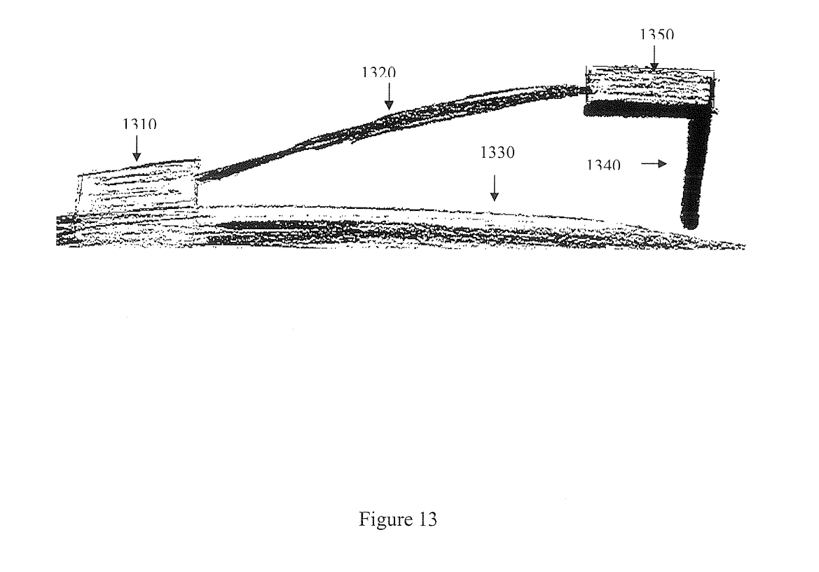

In one or more embodiments, the vibration sensor includes a sensing tip, wherein the tip increases detection sensitivity. In one or more embodiments, a metal tip is used to bend the vibration sensor, and the tip also acts as the contact point between the vibration sensor and the joint. Other materials, such as plastics, can be used for the tip. FIG. 13 is a schematic diagram illustrating the tip 1340, according to one or more embodiments. The metal tip is bent at about a 90° angle and attaches to an end of the piezoelectric sensor 1320 that is opposite to an end of the sensor that is attached to the limb 1330. Attachment element 1310, such as medical adhesive tape, is used to attach the vibration sensor 1320 to the limb 1330 of the subject. Attachment element 1350 attaches an opposite end of the sensor to the metal tip 1340. The metal tip can help stabilize the piezoelectric sensor and increases sensor sensitivity. In one or more embodiments, the metal tip has a rounded end. In one or more embodiments, the metal tip has an un-rounded tip.

In one or more embodiments, the vibration sensors are attached to the joint using attachment elements on one end of the sensor. Other attachment elements known in the art can be used, including a snap, a button, a fastener, a connector, or other interlocking fabric materials, such as Velcro, and tape, such as medical adhesive tape.

A plurality of sensors can be used in different locations near the joint. In some embodiments, two or three vibration sensors are used. In some embodiments, the vibration sensors are placed in various areas over the knee joint, including but not limited to the patellar tendon, patella, femur, fibula, tibia, anterior cruciate ligament, posterior cruciate ligament. In some embodiments, the vibration sensors are placed near the patellar tendon. In some embodiments, the vibration sensors are placed over the elbow joint, including but not limited to the radius, ulna, humerus, ulnar collateral ligament, radial collateral ligament, and annular ligament.

### Goniometer

As used herein, a goniometer is an instrument that measures an angle. Angle measurements indicate the range of motion of the joint, and thus the relative health of the joint. Various types of goniometers known in the art can be used in the instant devices and methods. One or more gonoiometers can be used in the instant devices and methods. In one or more embodiments, the flexible member contoured to fit over a joint on a limb of a subject acts as the goniometer, and the bracing member allows the limb to pivot. In one or more embodiments, the bracing member includes a flexible member contoured to fit over a joint on a limb of a subject, wherein the bracing member allows the limb to pivot; at least a first sensor operably connected to the bracing member, wherein the first sensor is a goniometer; and at least a second sensor operably connected to the bracing member, wherein the second sensor is configured to detect a vibration caused by joint movement.

In one or more embodiments, the goniometer is an electro-goniometer. In one or more embodiments, the goniometer is a commercial off the shelf (COTS) goniometer and includes a potentiometer. As disclosed herein, the electro-goniometer can be attached using various attachment elements known in the art, including a snap, a button, a fastener, a connector, tape, adhesive, and interlocking fabric materials, such as Velcro. In one or more embodiments, the joint sensor device includes a bracing member that is a sleeve with an inner surface and an outer surface. The goniometer can be integrated into the bracing member by being woven into the outer surface of the bracing member.

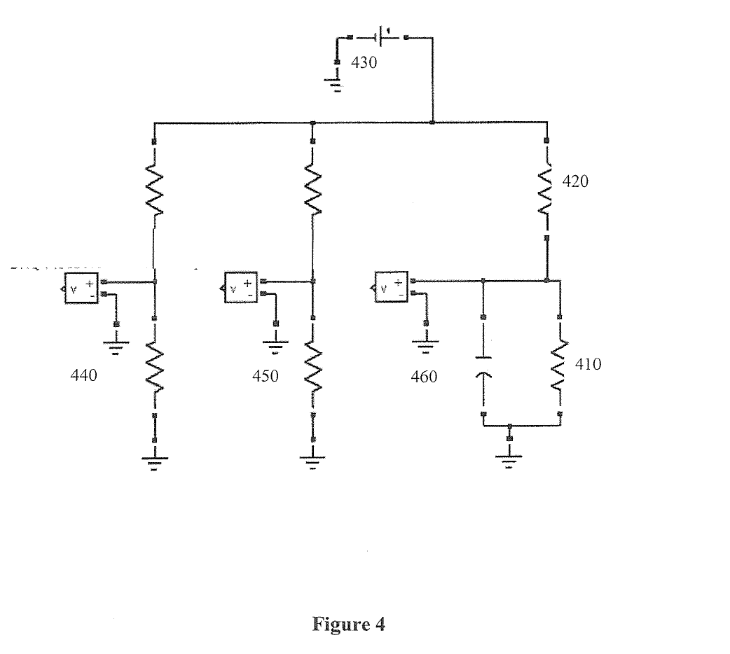

In one or more embodiments, the electro-goniometer was designed using a COTS mechanical goniometer and a 5K Ohm potentiometer. FIG. 4 is a schematic diagram of thermistors and a goniometer used in the disclosed devices. The joint sensor device was set up so that as the goniometer 410 moved to measure a specific angle from the extension and flexion of the limb, the dial of the potentiometer also moved, changing its resistance proportional to the amount the dial moved. The goniometer measurement is shown as 460. The potentiometer 410 was wired into a voltage divider circuit with a resister 420 (8150 Ohm). The voltage across the potentiometer is read into the DAQ (430) and is then processed in LabVIEW software through a low-pass filter to cancel out the noise from the wiring. FIG. 4 shows the thermistor measurements at 440 and 450.

The resistance of the potentiometer is calculated by the following equation:

Ri=Rs/((Vi/Vm)−l)

### Where,

Ri=Resistance of the Potentiometer

Rs=8140 Ohms

Vi=5.46V

Vm=Voltage Measurement across Ri from the DAQ.

The angle of the knee is then approximated using the following equation:

Angle=(5.3*((5*sqrt(2)*sqrt(311836382−61775*Ri)−45635)/2471)+45)−76

In another form, the formula for converting voltage to angle measurement is shown below:

1.4516((((R_goniomter−7357.6)/330.39)−1)*5)+22

This equation was derived through measured the voltage across the goniometer at known angles, comparing with the angle measurements displayed on the mechanical goniometer, and then calculating using a best fit line.

In one or more embodiments, the goniometer measures the angle of the limb's motion in more than one axis. In one or more embodiments, the limb can be moved in the x-direction with an angle that is 20°, about 30°, about 40°, about 50°, about 60°, about 70°, about 80°, about 90°, about 100°, or about 110°. In other embodiments, the slight angle variance in the y and z directions are also measured. Variations in the angle measurements can indicate a joint disease condition.

### Temperature Sensors

High temperature can indicate inflammation in the joint. In one or more embodiments, medical grade NTC Thermistors from GE (product number: MA100GGA). Any thermistors that have a fast thermal response time and a small tolerance in the human body temperature range (about 35.5-37.0° C.) can be used for measuring joint temperature. The thermistors are attached to the joint using various attachments elements, such as using medical adhesives. Temperature readings are taken in the localized joint area as well as at a point above or below the joint. Temperature readings taken at the localized joint area indicates the temperature of the joint. Temperature readings taken at points above or below the joint indicate a reference temperature of the skin not covering the joint.

A plurality of temperature sensors in different locations near the joint are contemplated. These temperature sensors can detect localized inflammation.

In one or more embodiments, the thermistor has a fast response time (for example, about 15 seconds in still air, about 2.0 seconds in still water) and an operating temperature range of 0° C. to 50° C. The tolerance of the thermistor in the temperature range of the human body is from about +/−0.05° C. to about +/−0.1° C.

Power is supplied to the thermistor from the DAQ's digital output. In one or more embodiments, the joint sensor devices include a voltage divider circuit using the thermistor, the 5.46V power source, and a 12K Ohm resister. The change in voltage is recorded through the DAQ as the thermistor changes temperature with the joint.

Calculations were made to convert the thermistor voltage into temperature. First, the resistance of the thermistor was calculated using the following equation:

Ri=Rs/((Vi/Vm)−l)

### Where,

Rt=Resistance of the Thermistor

Rs=12K Ohm Resistor

Vi=DAQ Supply Voltage=5.46V

Vm=Thermistor Voltage measured by the DAQ

The resistance was then converted to temperature using the following approximation equation:

T1*(B/ln(Rr/Ri))/((B/ln(Rr/Ri)−Tl))

### Where,

T1=Reference Temperature; This is a temperature in the desired range in which the resistance for the thermistor is known. For the calculations, this value was chosen to be 288K

B=Beta Coefficient for the Thermistor=3888.5

Rr=Reference Resistance at 288K=15.710K Ohms

Ri=Resistance of the Thermistor; Calculated above

### Software

Software used for building DAQs include Experimental Physics and Industrial control Systems (EPICS). Graphical programming environments include but are not limited to Visual C++, ladder logic, Visual Basic, and LabVIEW. In some embodiments, the DAQ is programmed with LabVIEW software for signal processing after data collection.

In one or more embodiments, the software package includes two main components: a relational database and a graphical user interface (GUI). In some embodiments, the software package communicates with the joint sensor device, through a USB connection with the Nl DAQ in the electronics box. This allows for real-time data collection and storage.

The database acts as the backend storage for the disclosed joint sensor devices. The database is capable of storing and maintaining new and existing patient information as well as quantitative data for the joints. This allows for long-term tracking of the patients joints and gives the medical professional the ability to compare results over a number of years, as described herein.

The GUI acts as the front end management tool to facilitate usage of the joint sensor device and handling of the back end storage for the operator. In some embodiments, the GUI has a simple-to-navigate interface that allows for quick and easy access to all patient records stored in the database. The user has the ability to view and compare multiple readings at one time.

In the instant disclosure, the database was developed using Microsoft SQL Server 2008 and is capable of storing patient and joint data. This data can be stored within the database in two tables, one containing all of the patient data and the other containing all of the device readings. The number of patients and number of readings contained in the database is only limited by the capacity of the host server. The software has the capability to hold unlimited entries.

The management of this database is exclusively handled using the Microsoft .NET Framework through a GUI developed in Microsoft Visual C#. The GUI has multiple different functions that allow the medical professional to navigate the database quickly and easily.

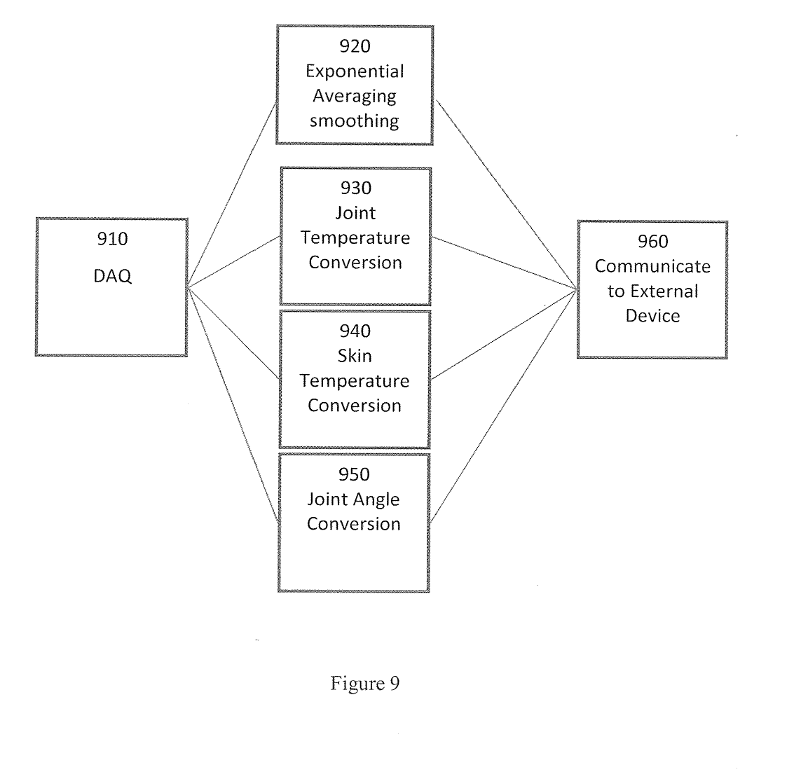

FIG. 9 is a schematic diagram illustrating the operations in the electronic conversions of sensor signals, according to one or more embodiments. In operation 910, the software acquires signal data the DAQ, which gathered data from the sensors placed on a joint of the limb of the subject. In some embodiments, the software, such as LabView Code, conducts operations 920, 930, 940, and 950 concurrently. In operation 920, the exponential averaging smoothing filter smoothes out noise from the acquired sensor signals, including the temperature, vibration, and angle signals. In operations 930 and 940, the software converts analog waveforms into digital values for processing; the voltages read from the temperature sensors are converted to degrees Celsius. In operation 950, the voltage read from the goniometer is converted to angle measurements. In operation 960, all sensor signal data is communicated to an external device. For example, the signal data is saved to a .csv file to be read by the GUI.

### Graphical User Interface

One or more embodiments of the graphical user interface are described herein.

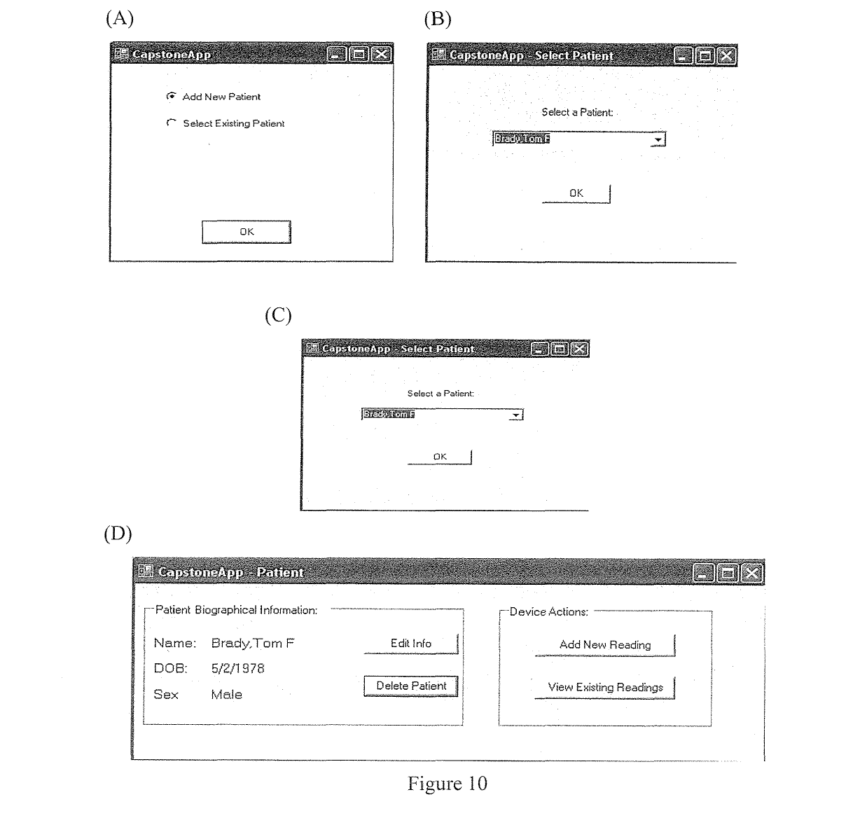

FIG. 10A is a diagram of the Main Screen. In the main screen of the GUI, the medical professional can select with a single click whether to add a new patient or to select an existing patient.

FIG. 10B is a diagram of the Add Patient screen. In this screen, the medical professional can add all of the required data for a new patient into the database. Fields include but are not limited to Last Name, First Name, Middle Initial, Date of Birth (DOB), and Sex.

FIG. 10C is a diagram of the Select Patient screen. The select patient screen allows the medical professional to access an existing patient's record through an easy to use drop down menu.

FIG. 10D is a diagram of the Main Patient Screen. The main patient screen displays, for example, the patients name, DOB, and sex of the patient. This screen also allows the medical professional to edit or delete patient information within the database. This screen is also where the management of patient readings can be accessed by selecting either Add New Reading or View Existing Readings.

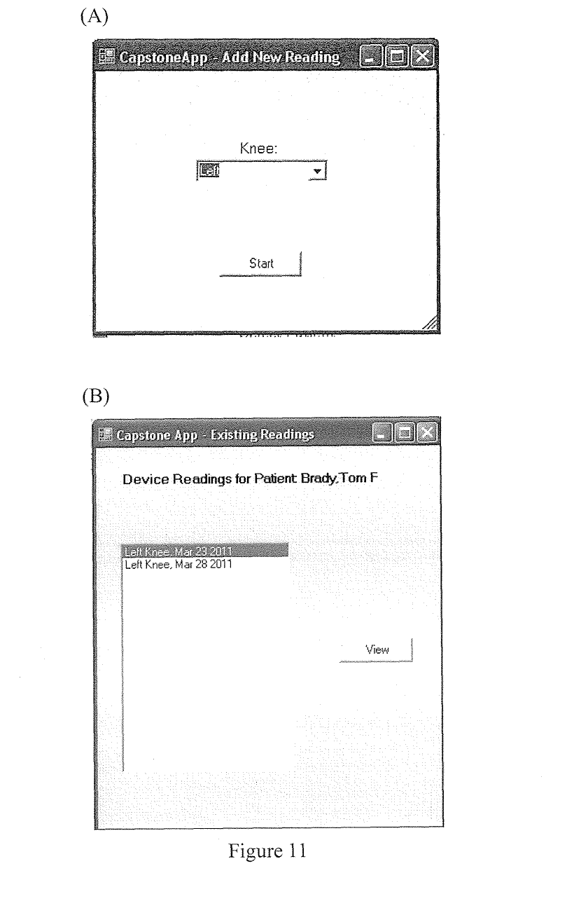

FIG. 11A is a diagram of the Add Reading Screen. From this screen, the medical professional can select to add new readings into the database for either the left or the right limb. This can be done by selecting either left or right from the drop down menu. The disclosed joint sensor device starts taking readings when the medical professional clicks on the ‘Start’ button on this screen. Once the joint sensor device finishes collecting data, the medical professional can choose to either save or discard the reading.

FIG. 11B is a diagram of the Existing Readings Screen. The existing readings screen allows the medical professional to select and view past data for a patient. The readings are sorted by date and left or right knee. The medical professional can choose to view multiple readings at one time.

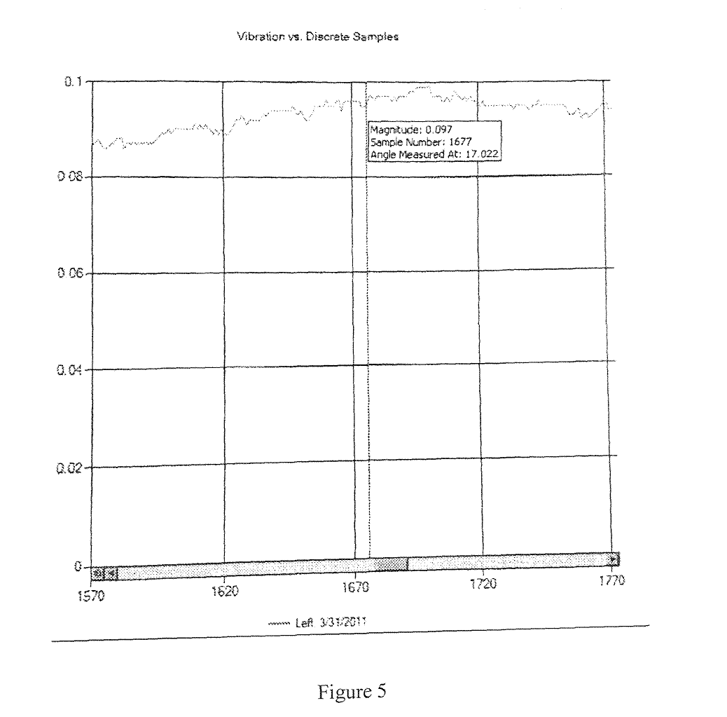

Each run for the joint sensor device can be displayed as a simple to read graph of vibration versus time. FIG. 5 is a graphical representation of data generated using an embodiment of the disclosed device. The x-axis represents sample numbers, and the y-axis represents the vibration magnitude (V). While viewing the data, a user can use a pointer feature to trace the graph and display the magnitude, angle measurement, and sample time of a data point. The pointer feature is illustrated in FIG. 5.

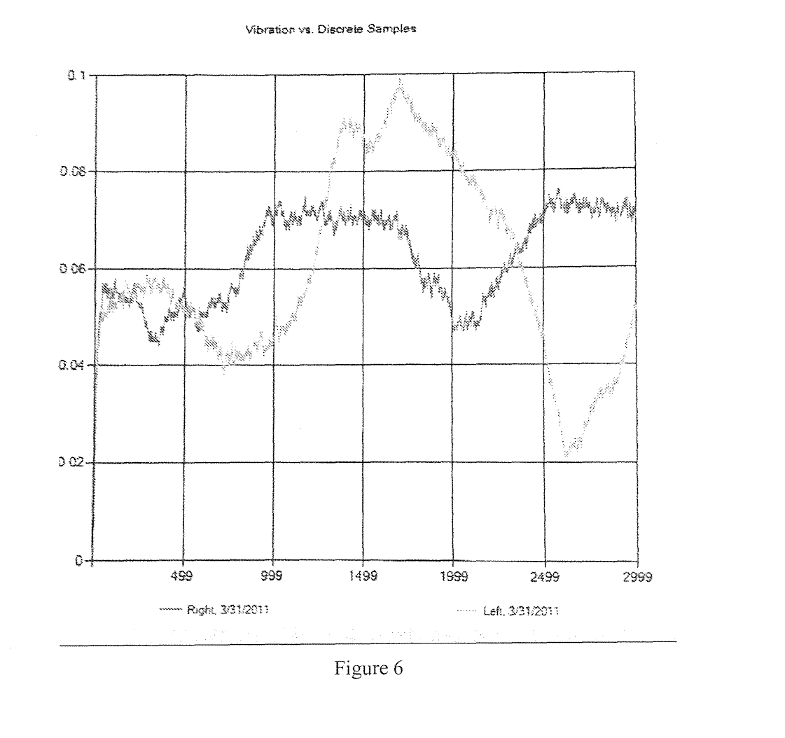

FIG. 6 is another graphical representation of vibration data generated using an embodiment of the disclosed device. The x-axis represents sample numbers, and the y-axis represents the vibration magnitude (V). FIG. 6 illustrates readings from the left knee and the right knee for the same individual compared on one graph.

### Methods

The disclosed methods allow for long-term data storage and data comparison regarding joint health. In one aspect, methods of monitoring joint health of a subject includes obtaining data using the joint sensor device disclosed herein; correlating the angle and vibration data; establishing a baseline data range over a period of time that reflects typical functioning of the joint of a subject; and comparing the baseline data range to test data from the subject to monitor joint health. In one or more embodiments, the temperature data are correlated with the angle and vibration data.

In one or more embodiments, the methods further include alerting the subject when the test data is significantly different from the baseline data range. For example, sudden spikes in the shape of the waveform would indicate a significant difference such that the subject is notified. The baseline data range is determined on a case-by-case basis and over a set period of time, ranging from weeks, months, to years.

In one or more embodiments, the methods further include diagnosing an arthropathy based on the comparison of the baseline data range to the test data.

Arthropathy may be localized to one joint, as with post-traumatic arthritis, or may affect more than one joint, as with osteoarthritis or rheumatoid arthritis. Neuropathic arthropathy refers to bone and joint changes that develop secondary to loss of sensation associated with various disorders, including diabetes, trauma, infection, pernicious anemia, spina bifida, or amyloidosis. Arthropathy is frequently associated with articular trauma, joint slippage (subluxation) and instability, or degenerative changes of the joint. The most common form of arthropathy is osteoarthritis, in which there is progressive loss of articular joint cartilage with reactive changes at the joint margins and subchondral bone, resulting in joint destruction.

The disclosed methods of tracking joint health are easy to operate and non-invasive. The disclosed methods quantifies joint health data and stores it in a database where the medical professional can easily recall and compare the data over time, effectively removing much of the human error associated with finding joint conditions. By tracking joint health over the long term, the medical professional can identify small changes in the joint, which can lead to earlier identification of health problems, and result in earlier and more effective treatment of a joint condition.

The disclosed devices and methods can be used during a periodic visit to a physician's office and can be an addition to the conventional physical check-up. In one or more embodiments, the device is placed on a joint, such as the knee, and the patient extends the knee a few times to record data to the joint sensor device. The location of the sensors can vary and be adjusted by the physician or the patient. In one embodiment, a patient swings his or her leg from a 90° angle to 0°, and then back to 90° angle at a particular speed as instructed by the physician. The angle data is stored in a database that is accessible by the physician for later review and analysis. The physician can then view and recall this data for years to help to identify and diagnose possible joint issues as a result of abnormalities in the data. The disclosed methods and devices allows a medical professional to quantify and compare data related to joint health, detect warnings signs of future joint problems, and also to diagnose a joint condition.

### EQUIVALENTS

As will be apparent to one of ordinary skill in the art from a reading of this disclosure, the disclosed subject matter can be embodied in forms other than those specifically disclosed above. The particular embodiments described above are, therefore, to be considered as illustrative and not restrictive. Those skilled in the art will recognize, or be able to ascertain, using no more than routine experimentation, numerous equivalents to the specific embodiments described herein. The scope of the invention is as set forth in the appended claims and equivalents thereof, rather than being limited to the examples contained in the foregoing description.

### EXAMPLES

### Example 1

One embodiment of the disclosed device was tested on five individuals. Wearing the disclosed joint sensor device, each individual rotated the angle of his knee from about a 90° angle to 0°, and then back to 90° angle at a particular speed. Each angle, joint temperature, and vibration measurement was taken from the same tendon on each individual's knee. The constant skin temperature was taken around each individual's ankle. All recorded data was then transferred to the GUI/Database and graphed on a computer.

Of the five members, all recorded data showed some variance from subject to subject. Thus, baseline data for an individual can be established on a case-by-case basis. The data was repeatable with similar results for each individual.

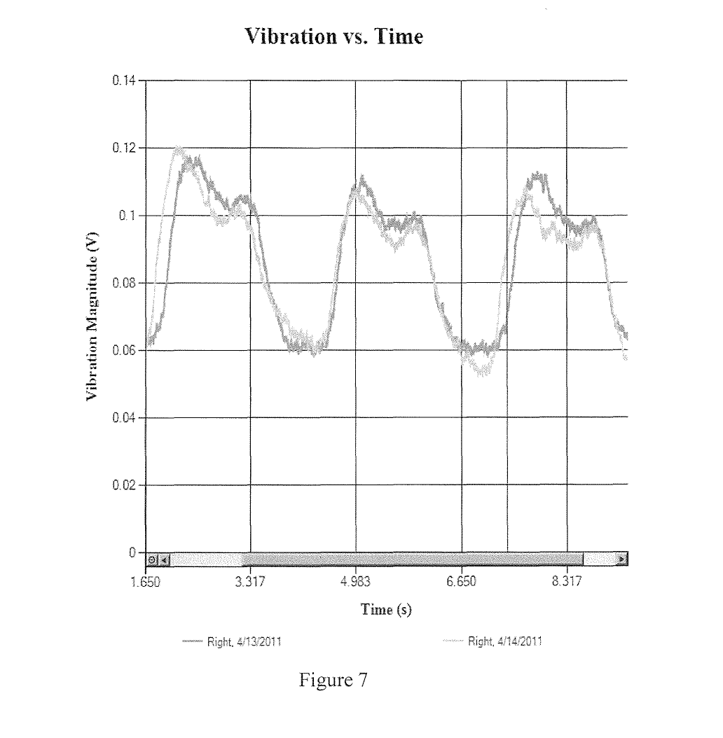

FIG. 7 is a graphical representation of vibration versus time data generated for two joints using the disclosed device. The data in FIG. 7 was generated from a relatively healthy knee, and can be used to establish a baseline range for an individual with healthy joints.

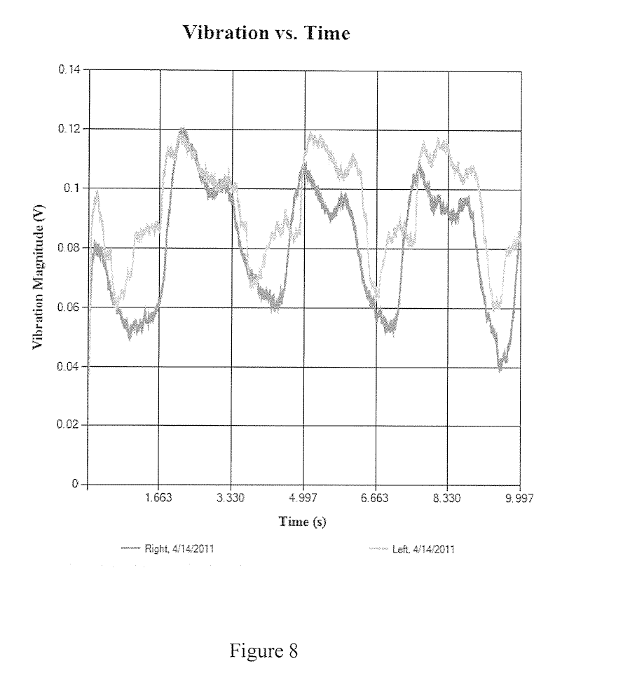

FIG. 8 is a graphical representation of vibration versus time data generated for the same individual who had experienced knee injury. Significant differences in waveform were observed between the healthy knee versus the injured knee.

### REFERENCES

[1] Y. T. Zhang et al., “Muscle Contraction Interference in Accelerated Vibroarthrography,” in Annu. Int. Conf. IEEE Engineering in Medicine and Biology Society, Vol. 12, No. 5, 1990, 1990©IEEE, doi: CH2936-3/90/0000-2150
[2] Rangaraj M. Rangayyan and Yunfeng Wu, “Screening of Knee-Joint Vibroarthrographic Signals Using Parameters of Activity and Radial-Basis Functions,” CCECE/CCGE1 May 5-7 2008 Niagara Falls. Canada, 2008©IEEE, doi:978-1-4244-1643-1/08
[3] S. Stanciu et al., “Vibroarthrography—a Possible Functional Non-invasive Method for Early Detection Damaged Cartilage Joint,” ROM. J. INTERN. MED., vol. 44, pp. 471-476, 2006.
[4] Sanjeev Tavathia et al., “Analysis of Knee Vibration Signals Using Linear Prediction,” IEEE Trans. Biomed. Eng., vol. 39, no. 9, pp. 959-970, September 1992.
[5] A. J. Collins and J. A. Cosh, “Temperature and biochemical studies of joint inflammation,” Ann. Rheum. Dis., vol. 29, pp. 386-392, 1970.
[6] National Instruments. (2008, March). NI USB-6008/6009 [Online]. Available: digital.ni.com/manuals.nsf/websearch/7781F8E689519ED786257411006FB09F
[7] General Electric. (2006). NTCType MA-Biomedical Chip Thermistors [Online]. Available: http://www.ge-mcs.com/en/temperature/medical-assemblies/ntc-type-ma.html
[8] Measurement Specialties. (2008, Oct. 13). Piezo Sensor-LDT Series [Online]. Available: http://www.meas-spec.com/product/t product.aspx?id=2484
[9] Hernandez, Michael J. Database Design for Mere Mortals: A Hands-On Guide to Relational Database Design. 2^{nd} ed. Boston, Mass.: Addison-Wesley, 2003.
[10] Forta, Ben. Sams Teach Yourself Microsoft SQL Server T-SQL in 10 Minutes. Indianapolis, Ind.: Sams Publishing, 2008.
[11] Mayo, Joe. “C# Station.” Internet: http://www.csharp-station.com/Default.aspx, 2010 [Apr. 3, 2011].
[12] Mitchell, Scott. “Microsoft Chart Controls, Visual Studio Automatic Code Snippets, And More.” Internet: http://msdn.microsoft.com/en-us/magazine/dd569763.aspx, 2010 [Apr. 3, 2011].

1. A joint sensor device, comprising:
(a) a bracing member contoured to fit over a joint on a limb of a subject, wherein the bracing member allows the limb to pivot;
(b) at least a first sensor operably connected to the bracing member, wherein the first sensor is configured to detect an angle of the joint;
(c) at least a second sensor operably connected to the bracing member, wherein the second sensor is configured to detect a vibration caused by joint movement; and
(d) a data communication device operably connected to the sensors, wherein the data communication device communicates signals from the sensors to an external device.

2. The joint sensor device of claim 1, wherein the bracing member acts as the first sensor.

3. The device of claim 1, further comprising at least a third sensor operably connected to the bracing member, wherein the third sensor is configured to detect a temperature.

4. The device of claim 3, wherein the third sensor is configured to detect the temperature of the joint and/or of a location away from the joint.

5. The device of claim 3, wherein the bracing member is a sleeve having an inner surface and an outer surface.

6. The device of claim 3, wherein the data communication device communicates sensor signals to an external device using a wireless communication protocol.

7. The device of claim 3, wherein the data communication device communicates sensor signals to an external device using a wire.

8. The device of claim 3, wherein the external device comprises memory and is configured to store and display the sensor signal data.

9. The device of claim 3, wherein the second sensor includes a sensing tip, wherein the tip increases detection sensitivity.

10. The device of claim 3, wherein the limb is the knee of the subject.

11. A bracing member, comprising:
(a) a flexible member contoured to fit over a joint on a limb of a subject, wherein the flexible member allows the limb to pivot;
(b) at least a first sensor operably connected to the flexible member, wherein the first sensor is configured to detect an angle of the joint; and
(c) at least a second sensor operably connected to the flexible member, wherein the second sensor is configured to detect a vibration caused by joint movement.

12. The bracing member of claim 11, wherein the flexible member acts as the first sensor.

13. The bracing member of claim 11, further comprising:
at least a third sensor operably connected to the flexible member, wherein the third sensor is configured to detect a temperature.

14. The bracing member of claim 13, wherein the third sensor is configured to detect the temperature of the joint and/or of a location away from the joint.

15. The bracing member of claim 14, wherein the flexible member is a sleeve having an inner surface and an outer surface.

16. The bracing member of claim 14, wherein the second sensor includes a sensing tip, wherein the tip increases detection sensitivity.

17. The bracing member of claim 14, wherein the limb is the knee of the subject.

18. A method of monitoring joint health of a subject, comprising:
(a) obtaining data using the joint sensor device of claim 1, wherein the angle and vibration data are correlated;
(b) establishing a baseline data range over a period of time that reflects typical functioning of the joint of a subject; and
(c) comparing the baseline data range to test data from the subject to monitor joint health.

19. The method of claim 18, wherein the temperature data are correlated with the angle and vibration data in step (a).

20. The method of claim 18, further comprising:
alerting the subject when the test data is significantly different from the baseline data range.

21. The method of claim 20, further comprising:
diagnosing an arthropathy based on the comparison of the baseline data range to the test data.
